(19) United States
(12) Patent Application Publication
(10) Pub. No.: US 2010/0262190 A1
(43) Pub. Date: Oct. 14, 2010

(54) SPINAL ROD TRANSLATION DEVICE

(75) Inventors: Rodney Ray BALLARD, Lakeland, TN (US); Jason Michael May, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/421,440

(22) Filed: Apr. 9, 2009

(51) Int. Cl. A61B 17/70; A61B 17/86; A61B 17/88

(52) U.S. Cl. 606/264; 606/301; 606/279

(57) ABSTRACT

A rod translation device is described that minimizes the wear between a spinal rod and a bone anchor in a spinal implant. In one embodiment, the rod translation device is a sleeve that is slid onto the outside diameter of the spinal rod. At least one compressible member, such as an O-ring, may be placed within the inside diameter of the sleeve to prevent direct contact of the outside diameter of the spinal rod to the inside diameter of the sleeve. Once the spinal rod is secured within the implant any translation of the spinal rod decreases wear debris between the outside diameter of the spinal rod and the pedicle screw.

### BACKGROUND OF THE INVENTION

The present invention generally relates to spinal fixation systems. More particularly, an embodiment of the invention relates to a spinal implant system for correction, fixation, and stabilization of the human spine. This may be to allow for the development of a solid spinal fusion or may be applied to non-fusion treatments of the spine.

Spinal fixation, such as lumbar sacral fusion and the correction of spinal deformities such as scoliotic curves, is a well known and frequently used medical procedure. Pedicle, lateral, and oblique mounting devices may be used to secure corrective spinal instrumentation to a portion of the spine that has been selected to be treated.

A spinal fixation system typically includes corrective spinal instrumentation that is attached to selected vertebrae of the spine by screws, hooks, and clamps. The corrective spinal instrumentation includes spinal rods or plates that are generally located parallel to the patient's back. The corrective spinal instrumentation may also include transverse connecting rods that extend between neighboring spinal rods. The spinal fixation systems are used to correct problems in the cervical, thoracic and lumbar portions of the spine, and are often installed posterior to the spine on opposite sides of the spine along the spinous process and adjacent to the transverse process.

In a typical implant scenario, a series of two or more pedicle screws may be inserted into two or more vertebra to be instrumented. A spinal rod may be secured to the pedicle screws using a set screw. The spinal rod is placed within a connecting device that links the rod and the pedicle screw. Thereafter, the set screw and all the other connections are tightened. In this way, a rigid supporting structure is fixed to the vertebrae, with the rod providing the support that promotes correction or healing of the vertebral malformation injury by keeping the vertebrae in a particular position.

A multitude of spinal fixation systems exist; however, the systems can be difficult to assemble and secure and can cause tissue irritation and/or damage to surrounding area, as well as wear on the spinal rod as it translates in the spinal implant.

Thus, a need exists for a device that allows translation of a spinal rod with respect to a spinal implant with minimal wear.

### SUMMARY OF THE INVENTION

In accordance with the present invention, a spinal fixation system is provided that reduces the wear of a spinal rod as it translates in a spinal implant. The device allows translation of the spinal rod with respect to a bone anchor with minimal wear debris. The device attaches to the rod and prevents a clamping force, due to the torque of the set screw, from acting on the rod.

In one embodiment, the device can be a cannulated cylinder, or sleeve, through which the rod is allowed to translate. The length of the cylinder can be long enough to accommodate engagement to the pedicle screw and set screw.

In another embodiment, the device can also have one or more multiple compressible members retained in its inner diameter to prevent the rod from contacting the cylinder. The compressible member prevents metal to metal contact thereby reducing wear. In yet another embodiment, the compressible member in the device is an 0-ring.

In general, the present invention relates to a rod translation sleeve that attaches to a spinal rod and allows translation of the spinal rod with respect to a bone anchor with minimal wear to the rod. The device attaches to the rod and prevents the clamping force due to the torque of the set screw from acting on the rod. The device can be a cannulated cylinder, or sleeve, through which the rod is allowed to translate. The length of the sleeve can be long enough to accommodate encasement to the pedicle screw and set screw. Multiple compressible members may be retained within the cylinder to prevent the rod from contacting the cylinder.

Further embodiments, forms, features, aspects, benefits, objects and advantages of the present invention will become apparent from the detailed description and figures provided herewith.

### BRIEF DESCRIPTION OF THE FIGURES

FIG. 1 is a perspective view of an unassembled version of an embodiment of the invention.

FIG. 2 is an assembled version of the embodiment described in FIG. 1.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation on the scope of the invention is hereby intended, and that alterations and further modifications to illustrated devices and/or further applications of the principles of the invention as illustrated herein are contemplated as would normally occur to one skilled in the art to which the invention relates.

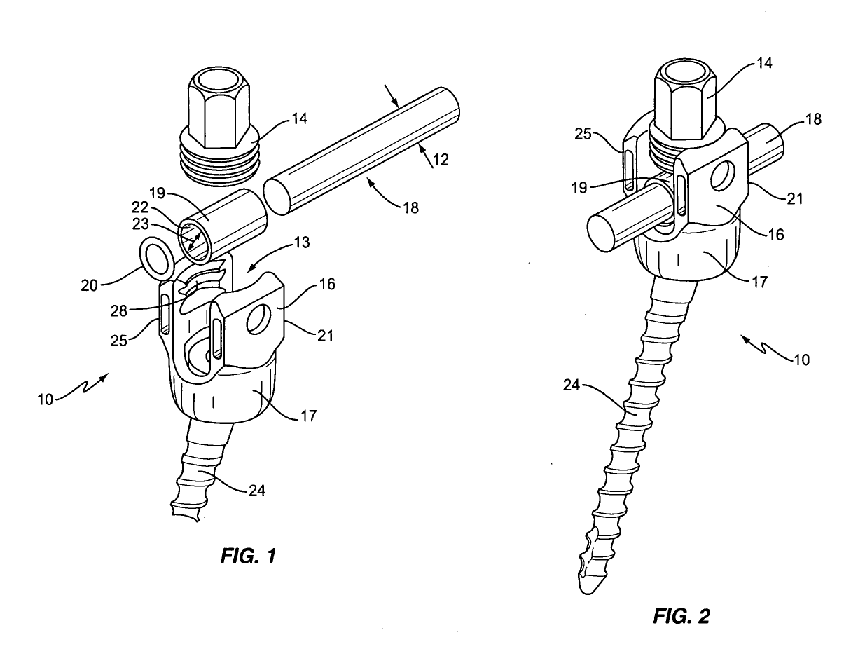

FIGS. 1 and 2 describe an embodiment, unassembled and assembled, of the invention, respectively. In FIG. 1, a spinal connector 10 comprising a pedicle screw 24 with a tulip shaped top portion 16 is illustrated. The tulip shaped top portion 16 of the pedicle screw 24 comprises a top portion 17 with a first arm 21 and a second arm 25. Together, the first and second arms 21,25 form a substantially U-shaped bore 13 into which a spinal rod 18 may be axially positioned. The tulip shaped top portion 16 may include a thread-form 28 to accept a set screw 14. The tulip shaped top portion 16 retains the spinal rod 18 within the U-shaped bore 13. The spinal rod 18 may be composed of an implant grade material such as stainless steel, titanium, PEEK (polyetherether ketone), etc. A cannulated cylinder, or sleeve 19, is slid onto the outside diameter 12 of the spinal rod 18 before the spinal rod 18 is positioned within the tulip shaped top portion 16 of the pedicle screw 24. The sleeve 19 may be composed of an implant grade metal, (stainless steel, titanium) or a polymeric material, such as PEEK. It is important to insure that the materials used for the spinal connector 10 and the cannulated cylinder are biocompatible and galvanically suitable.

The sleeve 19 is sized or cut to fit within the confines of the tulip head portion 16 of the pedicle screw 24. The pedicle screw 24 could also be substituted with a fixed angle screw (not shown) or a hook (not shown). Any spinal connector that utilizes a bore to capture a spinal rod 18 could be used.

As an option, a polymeric material can be added within the sleeve 19. In FIG. 1, the polymeric material comprises at least one compressible member added within the sleeve 19 to prevent metal to metal contact of the spinal rod 19 to the inside surface 22 of the sleeve 19. In FIG. 1, the compressible member is an O-ring 20 that is sized to be retained within the inner diameter 22 of the sleeve 19 and between the outside diameter 12 of the spinal rod 18. Hence, the O-ring 20 is between the inside diameter 22 of the sleeve 19 and the outside diameter 12 of the spinal rod 18. The O-ring 20 may be composed of an elastomeric/polymeric material such as an implant grade elastomer or PEEK.

The set screw 14 may then be threaded into the tulip shaped top portion 16 of the spinal implant 10 to secure the spinal rod 18. As the set screw 14 is screwed into the tulip shaped top portion 17 it contacts the sleeve 19. This contact reduces movement, and tightens the sleeve 19 within the tulip shaped top portion 16. As a consequence to the screwing of the set screw 14, the O-ring 20 compresses on the spinal rod 18. Further screwing of the set screw 14 increases the compression of the O-ring on the spinal rod 18 securing the spinal rod 18 in a slideable engagement with the sleeve 19. Translation of the spinal rod 18 within the sleeve 19 is still possible, but wear debris produced from metal-to-metal contact between the spinal rod 18 and the tulip shaped top portion 17 is reduced.

Any theory, mechanism of operation, proof, or finding stated herein is meant to further enhance understanding of the present invention, and is not intended to make the present invention in any way dependent upon such theory, mechanism of operation, proof or finding. It should be understood that while the use of the word preferable, preferably or preferred in the description above indicates that the feature so described may be more desirable, it nonetheless may not be necessary, and embodiments lacking the same may be contemplated as within the scope of the application, that scope being defined by the claims that follow. In reading the claims, it is intended that when words such as “a”, “an”, “at least one”, and “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

While the application has been illustrated and described in detail in the drawings and foregoing description, the same is to be considered as illustrative and not restrictive in character, it being understood that only the select embodiments have been shown and described and that all changes, modifications and equivalents that come within the spirit of the invention as defined herein or by any of the following claims are desired to be protected.

What is claimed is:

1. A device that allows translation of a spinal rod, comprising:
a spinal connector that accommodates capture of the spinal rod;
a spinal rod securing device that secures the spinal rod in the spinal connector; and
a cannulated cylinder that slideably engages the spinal rod and is affixed between the spinal rod securing device and a spinal connector within the confines of the spinal connector.

2. The device of claim 1, further comprising an polymeric material that is retained in the cannulated cylinder and wherein the spinal rod passes through the polymeric material.

3. The device of claim 2, wherein the polymeric material is a compressible member.

4. The device of claim 3, wherein at least one compressible member is retained in the cannulated cylinder and wherein the spinal rod passes through the compressible member.

5. The device of claim 1, wherein the spinal connector comprises a pedicle screw with a rod capturing portion and a bore to allow longitudinal placement of the spinal rod.

6. The device of claim 5, wherein the spinal rod securing device comprises a set screw.

7. The device of claim 1, wherein the cannulated cylinder comprises a sleeve of implant grade material.

8. The device of claim 7, wherein the implant grade material is a metal.

9. The device of claim 7, wherein the implant grade material is a polymeric material.

10. The device of claim 1, wherein the spinal rod is an implant grade metal.

11. The device of claim 1, wherein the spinal rod is a polymeric material.

12. The device of claim 4, wherein the compressible member comprises a ring of polymeric material.

13. The device of claim 12, wherein the ring is an O-ring.

14. A method for spinal rod translation with respect to a bone anchor, wherein the bone anchor comprises a spinal connector with a pedicle screw and a tulip shaped top portion that captures the spinal rod in a longitudinal orientation, comprising:
sliding a sleeve onto the outside diameter of the spinal rod;
capturing the sleeve on the spinal rod within the tulip shaped top portion of the spinal connector; and
securing the sleeve in the tulip shaped top portion of the spinal connector with a set screw.

15. The method of claim 14, further comprising placing at least one O-ring on the outside diameter of the spinal rod and within the sleeve at the tulip shaped top portion of the spinal connector.

16. A spinal implant, comprising:
a tulip head pedicle screw;
a spinal rod longitudinally positioned within the tulip head pedicle screw, the spinal rod having an outside diameter;
a sleeve of implant grade material slideably engaging the outside diameter of the spinal rod, the sleeve having an inside diameter;
a compressible O-ring retained within the inside diameter of the sleeve; and
a set screw engaging the spinal rod within the tulip head pedicle screw by compressing on the sleeve.
